(19) United States
(12) Patent Application Publication
(10) Pub. No.: US 2009/0203607 A1
(43) Pub. Date: Aug. 13, 2009

(54) Method And Compositions For Simultaneously Regulating Memory And Mood

(76) Inventors: Evian Gordon, Vaucluse (AU); Lea Williams, Broadway (AU)

Correspondence Address: ST. ONGE STEWARD JOHNSTON & REENS, LLC, 986 BEDFORD STREET, STAMFORD, CT, 06905-5619, US

(21) Appl. No.: 12/371,248

(22) Filed: Feb. 13, 2009

Related U.S. Application Data
(63) Continuation of application No. PCT/AU2007/001140, filed on Aug. 14, 2007.
(60) Provisional application No. 60/837,719, filed on Aug. 14, 2006.

(30) Foreign Application Priority Data
Aug. 14, 2006 (AU) 2006904420

(51) Int. Cl. A61K 38/16; A61K 31/5375; A61K 31/381; A61K 31/137; A61P 25/28

(52) U.S. Cl. 514/12; 514/239.2; 514/438; 514/649

(57) ABSTRACT

The present invention is concerned with methods and compositions for simultaneously treating or modulating memory and mood within the same individual.

### CROSS-REFERENCE TO RELATED APPLICATIONS

The present application is a continuation of pending International patent application PCT/AU2007/001140 filed on Aug. 14, 2007 which designates the United States and claims priority from Australian patent application 2006904420 filed on Aug. 14, 2006 and U.S. Provisional Patent Application Ser. No. 60/837,719 filed on Aug. 14, 2006. All prior applications are herein incorporated by reference in their entirety.

### FIELD OF THE INVENTION

The present invention belongs to the fields of neuropharmacology, medicine and medicinal chemistry, and provides methods and compositions for simultaneously influencing memory and negative mood within the same individual.

The therapeutic composition can treat both memory and mood within the same individual and consists of at least a cholinesterase inhibitor and or a method to increase acetylcholine and at least an antidepressant. In one form, the antidepressant will be selected from compounds that increase the availability of serotonin, and in the alternate form, from compounds that increase availability of nor-epinephrine. Stimulation of serotonin will also stimulate brain-derived neurotropic factor (BDNF) which will influence both memory and mood. A BDNF compound and or a BDNF stimulator can also be added to the inventive drug combination of a cholinesterase inhibitor and a serotonin and or nor-epinephrine re-uptake blocker. The insights for this invention drew upon converging evidence in the Brain Resource International Database.

### BACKGROUND OF THE INVENTION

Patients taking memory enhancing agents typically also experience negative mood, which is not alleviated by these agents. Negative mood problems will compound memory problems. Thus, it is important to alleviate negative mood in patients with memory problems, but these patients do not gain mood benefit from existing memory agents.

Conversely, patients with mood disorders typically have modifications in memory that result in functional memory deficits in affected individuals. Mood modifying anti-depressants do not target these memory deficits.

Memory modifying agents usually only produce a response for chronic dosages.

Similarly, mood modifying anti-depressants also usually only produce a response to chronic dosages, and may only work in some patients and/or the response may be suboptimal to normalize behavior. Thus, patients do not gain immediate benefit from an acute dosage of memory agents or anti-depressants.

Not all patients will be assisted by memory agents or anti-depressants even after prolonged therapy.

The likely reason why memory agents and antidepressants do not always result in a benefit to patients for memory and negative mood, is because many neurochemical systems are inter-linked, and these other neuro-chemical systems and are also likely to be dysfunctional.

The inventor has identified the cholinergic and serotonergic systems (which are modulated by the neurotrophic growth factor BDNF as those amenable to modification in order to regulate both memory and mood in the same individual. Noradrenergic systems are an alternate pathway to modifying negative mood, and are also moderated by BDNF.

Based on a lack of efficacious drugs to treat both memory and mood in the same individual there is an immediate need to have a drug formulation and/or combination to modify both memory and mood within the same patient in order to improve, restore and/or prevent decline in cognitive function. This is particularly important for the aging population where memory problems are on the increase. Negative mood will act to exacerbate memory problems in these individuals.

The inventor has identified a way to improve both memory and mood in the same individual by using combinational drug therapy. It is an object of the present invention to overcome or ameliorate at least one of the disadvantages of the prior art, or to provide a useful alternative.

### SUMMARY OF THE INVENTION

The present invention relates to compositions and methods for supplementing or complementing natural central nervous system neurotransmitter activity to optimize global brain function, particularly in disorders associated with combined memory and mood impairment and or disorders that may influence both memory and negative mood.

The present invention provides novel pharmaceutical compositions having biological activity for the modulation and/or stabilization of mood and memory within the same individual. The present invention further provides methods for treating or preventing diseases of the central nervous system that result in mood and memory problems by using the novel compositions.

In accordance with an aspect of present invention, novel pharmaceutical compositions are provided.

The compositions of the present invention combine at least one memory enhancing agent and at least one antidepressant. More specifically, the compositions of the present invention combine at least one active agent that increases acetylcholine and at least one active agent that is a serotonin uptake inhibitor or nor-epinephrine uptake inhibitor.

The compositions of the present invention are considered to be particularly effective in the prophylactic or therapeutic treatment of combined memory and mood deficits or disorder within an individual.

Thus, according to a first aspect there is provided a composition comprising a pharmaceutically effective amount of one or more memory enhancing agents and a pharmaceutically effective amount of one or more antidepressants.

According to a second aspect there is provided a composition comprising a pharmaceutically effective amount of one or more antidepressants and a pharmaceutically effective amount of one or more cholinesterase inhibitors.

According to a third aspect there is provided a composition comprising a pharmaceutically effective amount of one or more cholinesterase inhibitors and a pharmaceutically effective amount of one or more serotonin uptake or reuptake inhibitors.

According to a fourth aspect there is provided a composition comprising a pharmaceutically effective amount of one or more anti-cholinesterase agents and a pharmaceutically effective amount of one or more norepinephrine reuptake inhibitors

According to a fifth aspect there is provided a composition comprising a pharmaceutically effective amount of one or more anti-cholinesterase agents and a pharmaceutically effective amount of one or more serotonin uptake or reuptake inhibitors and/or one or more nor-adrenaline reuptake inhibitors.

According to a sixth aspect there is provided a composition comprising a pharmaceutically effective amount of one or more anti-cholinesterase agents and a pharmaceutically effective amount of one or more serotonin uptake or reuptake inhibitors and/or one or more nor-adrenaline reuptake inhibitors, and a stimulator and/or inducer of BDNF expression and/or activity.

According to a seventh aspect there is provided a composition comprising: (a) a pharmaceutically effective amount of one or more serotonin reuptake inhibitors and/or one or more nor-adrenaline reuptake inhibitors, and (b) a pharmaceutically effective amount of one or more anticholinesterase agents, and (c) a stimulator of BDNF expression and/or activity.

According to an eighth aspect there is provided a composition comprising: (a) a pharmaceutically effective amount of one or more anti-cholinesterase agents, and (b) a pharmaceutically effective amount of one or more serotonin uptake or reuptake inhibitors and/or one or more nor-adrenaline reuptake inhibitors, wherein the composition up-regulates BDNF expression.

According to a ninth aspect there is provided a composition comprising: (a) a pharmaceutically effective amount of one or more anti-cholinesterase agents, and (b) a pharmaceutically effective amount of one or more serotonin uptake or reuptake inhibitors and/or one or more nor-adrenaline reuptake inhibitors.

According to a tenth aspect there is provided a composition comprising: (a) a pharmaceutically effective amount of one or more anti-cholinesterase agents; and (b) a pharmaceutically effective amount of one or more serotonin uptake or reuptake inhibitors and/or one or more noradrenaline reuptake inhibitors; and (c) a 5HT1a agonist or antagonist.

According to an eleventh aspect there is provided a composition comprising: (a) a pharmaceutically effective amount of one or more anti-cholinesterase agents; and (b) a pharmaceutically effective amount of one or more serotonin uptake or reuptake inhibitors and/or one or more noradrenaline reuptake inhibitors; and (c) a 5HT1a agonist or antagonist and (d) a compound that increases BDNF expression or activity.

According to a twelfth aspect there is provided a composition comprising a pharmaceutically effective amount of one or more anti-cholinesterase agents and one or more of the following: (a) a 5HT1 agonist or antagonist, (b) a compound that increases BDNF expression or activity, (c) an anti-depressant.

All compositions of the present invention may optionally include a stimulator or enhancer of BDNF expression and/or activity such as for example an estrogen, an estrogen derivative or a phyto-estrogen. Such agents may up-regulated BDNF expression. It will be understood that the pharmaceutically active agents used in compositions of the present invention may include any pharmaceutically effective salt of the active agent. The compositions may optionally comprise one or more pharmaceutically acceptable excipients and/or solvents.

According to a thirteenth aspect there is provided a method of prophylactic or therapeutic treatment of memory and mood in a subject comprising the administration of a therapeutically effective amount of at least one memory enhancing agent and at least one antidepressant to a subject requiring such treatment.

According to a fourteenth aspect there is provided a method of prophylactic or therapeutic treatment of memory and mood comprising the administration of a therapeutically effective amount of at least one antidepressant and at least one cholinesterase inhibitor to a subject requiring such treatment.

According to a fifteenth aspect there is provided a method of prophylactic or therapeutic treatment of memory and mood comprising the administration of a therapeutically effective amount of at least one cholinesterase inhibitor and at least one serotonin uptake or reuptake inhibitor to a subject requiring such treatment.

According to a sixteenth aspect there is provided a method of prophylactic or therapeutic treatment of memory and mood comprising the administration of a therapeutically effective amount of at least one anti-cholinesterase agent and at least one norepinephrine reuptake inhibitor to a subject requiring such treatment.

According to a seventeenth aspect there is provided a method of prophylactic or therapeutic treatment of memory and mood comprising the administration of a therapeutically effective amount of at least one anti-cholinesterase agent and at least one serotonin uptake or reuptake inhibitor and/or one or more nor-adrenaline reuptake inhibitor to a subject requiring such treatment.

According to an eighteenth aspect there is provided a method of prophylactic or therapeutic treatment of memory and mood comprising the administration of a therapeutically effective amount of at least one anti-cholinesterase agent and at least one serotonin uptake or reuptake inhibitor and/or at least one nor-adrenaline reuptake inhibitor, and a stimulator and/or inducer of BDNF expression and/or activity to a subject requiring such treatment.

According to a nineteenth aspect there is provided a method of prophylactic or therapeutic treatment of memory and mood comprising the administration of a therapeutically effective amount of at least one serotonin reuptake inhibitor and/or at least one nor-adrenaline reuptake inhibitor, at least one anti-cholinesterase agent, and a stimulator of BDNF expression and/or activity to a subject requiring such treatment.

According to a twentieth aspect there is provided a method of prophylactic or therapeutic treatment of memory and mood comprising the administration of a therapeutically effective amount of at least one anti-cholinesterase agent, at least one serotonin uptake or reuptake inhibitors and/or at least one nor-adrenaline reuptake inhibitor to a subject requiring such treatment, wherein BDNF expression is up-regulated via an anti-depressant.

According to a twenty first aspect there is provided a method of prophylactic or therapeutic treatment of memory and mood comprising the administration of a therapeutically effective amount of at least one anti-cholinesterase agent, at least one serotonin uptake or reuptake inhibitor and/or at least one nor-adrenaline reuptake inhibitor to a subject requiring such treatment.

According to a twenty second aspect there is provided a method of prophylactic or therapeutic treatment of memory and mood comprising the administration of a therapeutically effective amount of at least one anti-cholinesterase agent, at least one serotonin uptake or reuptake inhibitor and/or at least one noradrenaline reuptake inhibitor; and a 5HT1a agonist or antagonist to a subject requiring such treatment.

According to a twenty third aspect there is provided a method of prophylactic or therapeutic treatment of memory and mood comprising the administration of a therapeutically effective amount of at least one anti-cholinesterase agent, at least one serotonin uptake or reuptake inhibitor and/or at least one noradrenaline reuptake inhibitor, a 5HT1a agonist or antagonist and a compound that increases BDNF expression or activity to a subject requiring such treatment.

According to a twenty fourth aspect there is provided a method of prophylactic or therapeutic treatment of memory and mood comprising the administration of a therapeutically effective amount of at least one anti-cholinesterase agent and one or more of the following: (a) a 5HT1 agonist or antagonist, (b) a compound that increases BDNF expression or activity, (c) an anti-depressant, to a subject requiring such treatment.

Compounds that stimulate, enhance or induce BDNF expression or activity can be selected for example from estrogens, estrogen derivatives or phyto-estrogens, which can up-regulate expression of BDNF.

It will be understood from the disclosure provided herein that different active agents may be administered as a combination formulation but may also be administered separately by way of co-administration either simultaneously or sequentially.

Although the methods for treating or preventing memory and mood disorders of the central nervous system as described herein are primarily intended for use in humans, other mammals may benefit from such treatment and are contemplated herein. In most instances the disease or disorder to be treated is neurological and or psychiatric condition causing combined memory and mood deficit and/or change that requires modulation, improvement, stabilisation, cognitive enhancement, cognitive stabilisation, neurological treatment or prevention of decline, or a combination thereof.

Other objects of the present invention will readily be apparent to those skilled in the art as reference is made to the detailed description of the preferred embodiment.

### BRIEF DESCRIPTION OF THE DRAWINGS

FIG. 1 is a summary of the BRC Methodology used in Gene-Brain-Behavior.

FIG. 2 is an overview of Gene-Brain-Behavior test results.

### DETAILED DESCRIPTION OF THE INVENTION

In describing the preferred embodiment, certain terminology will be utilized for the sake of clarity. Such terminology is intended to encompass the recited embodiment, as well as all technical equivalents which operate in a similar manner for a similar purpose to achieve a similar result.

In certain embodiments of the present invention novel compositions of different chemical entities is used to treat components of a disorder that affect mood and memory, such that memory and mood are improved and more specifically, the first chemical entity being a cholinesterase inhibitor and the second chemical entity being a serotonin re-uptake inhibitor and or a nor-epinephrine reuptake inhibitor.

It will be recognized that a mood disorder may result in a memory disorder at any point in time and or a memory disorder may influence the development of a mood disorder at any point in time, additionally many disorders may result in both a memory and mood disorder occurring at the same time or not. Additionally it will be appreciated that mood and memory may be one of many components to a disorder and the disorder may contain other symptoms and or signs that are can be influenced by the therapeutic combination or are not influenced by the therapeutic combination.

It will also be recognized that improvement in memory and mood may be determined subjectively and objectively using either patient feedback and or via the use of external examination, for example using clinical tools that provide validated measures of mood and memory and or functional information on the performance of brain regions that regulate either mood and or memory.

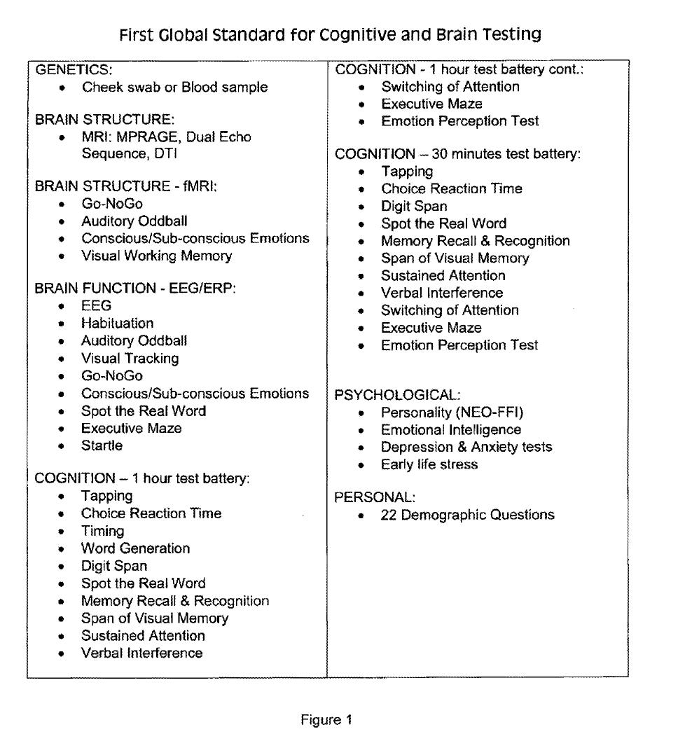

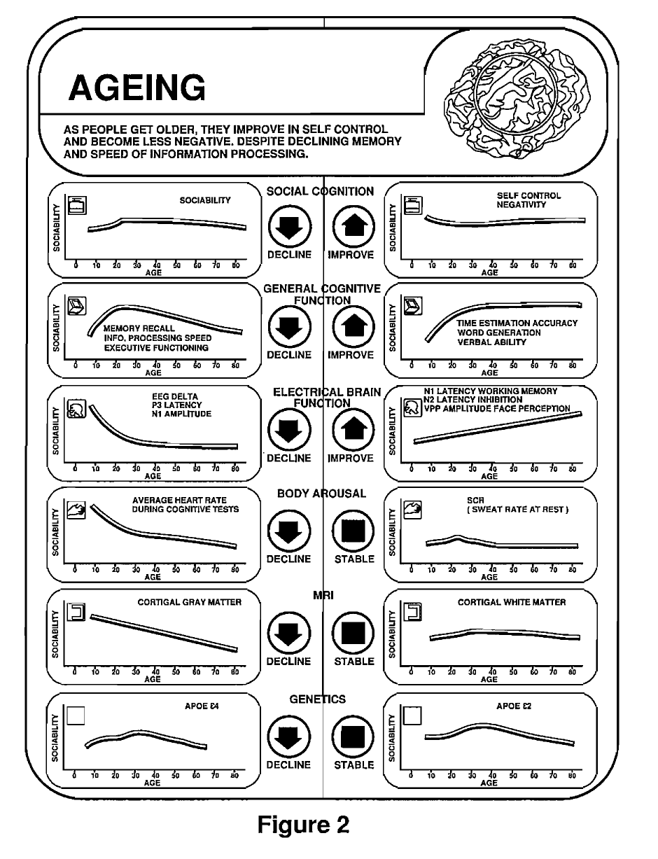

Insight for this combination therapy has drawn upon findings from the Brain Resource International Database—which has set up a global standard for Gene-Brain-Behavior testing (see BRC Methodology summary page in FIG. 1). Findings include memory decline with age, but relative preservation of emotion processing (see 1 page overview in FIG. 2), plus memory plus mood problems in a number of disorders. Detailed disclosure of the relevant methodology is provided in U.S. patent application Ser. No. 11/091,048, incorporated in its entirety herein by reference. It will be appreciated by those skilled in the science that memory and mood may be influenced not in the same direction in the one patient to gain a positive outcome for that patient via the administration of the therapeutic composition.

For example, it will also be appreciated that it is not necessary for there to be an improvement in either memory or mood for there to be a therapeutic effect for the combination therapy described in the present invention. That is the use of at least one anti-depressant and at least one cholinergic drug may be equally effective in slowing down the worsening of mood and or memory and thereby be effective, while not actually causing an improvement in either mood or memory.

There may also be an improvement in memory and/or mood and no change to either memory or mood in the other direction, while the disorder may have adversely caused progressive decline in both memory and mood over time. In these circumstances it will be appreciated that there is a therapeutic outcome in both mood and memory.

Hence with regard to the present invention there is scope to modulate mood and memory such that it is favourable to the patient with respect to the disorder causing problems and/or potential future problems to mood and memory.

The inventive compositions to treat memory and mood can also be used to treat any of the diseases or disorders of the central nervous system and/or the systemic body that can influence both mood and memory within the same individual. Such diseases and disorders are defined in The Diagnostic and Statistical Manual of Mental Disorders-IV (DSM-IV) (American Psychiatric Association (1995). To the extent necessary for completion, the contents of this reference and all of the defined diseases or disorders are expressly incorporated by herein by reference. Representative diseases or disorders include, but are not limited to, the following: mild cognitive impairment, Alzheimer's disease, the subtypes of Dementia, obesity, depression, bipolar disorder, schizophrenia, a stress related disease (e.g. general anxiety disorder), panic disorder, a phobia, obsessive compulsive disorder, post-traumatic-stress syndrome, immune system depression, incontinence, a stress induced problem with the urinary, gastrointestinal or cardiovascular system (e.g., stress incontinence), neurodegenerative disorders, autism, chemotherapy-induced vomiting, hypertension, migraine headaches, cluster headaches, sexual dysfunction in a mammal (e.g. a human), addictive disorder and withdrawal syndrome, an adjustment disorder, an age-associated learning and mental disorder, anorexia nervosa, apathy, an attention-deficit disorder due to general medical conditions, attention-deficit hyperactivity disorder, bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, a movement disorder (e.g., Tourette's syndrome), oppositional defiant disorder, a pain disorder, peripheral neuropathy, post-traumatic stress disorder, premenstrual dysphoric disorder, a psychotic disorder, seasonal affective disorder, a sleep disorder, a specific developmental disorder, and selective serotonin reuptake inhibition (SSRI) “poop out” syndrome. Treatment of the above diseases or disorders is accomplished by delivering a therapeutically effective amount of the inventive composition to a mammal such that mood and memory is influenced and or treated. In most cases this will be a human being, but treatment of food animals (e.g., livestock and poultry) and companion animals (e.g., dogs, cats and horses) is expressly covered herein.

The inventor has identified certain drug combination as being beneficial for both memory and mood within the same individual that is the use of a least one cholinesterase inhibitor and at least one antidepressant. Additionally the inventor has identified that there can be a further benefit to the patient via the addition of a compound that interacts with these neuro-chemical systems such as a compound that enhances BDNF function or the presence of increased and/or optimal BDNF activity.

The inventor has identified that also the drug combination of an acetylcholine inhibitor plus an antidepressant 5HT1 agonist or antagonist (and other 5HT receptors) will be effective in modifying memory and mood within the same affected individual. Without wishing to be bound by theory or any particular mechanism of action, it is believed that the presence of an antidepressant SSRI for serotonin will inhibit serotonin reuptake. Since this action takes place at the nerve ending of each serotonergic neuron, neurotransmission due to serotonin is enhanced. The inhibition of rapid serotonin reuptake by SSRI, however, takes place also in serotonergic neuron cell bodies and dendrites, which are present in the raphe nucleus. Therefore, the negative feedback through autoreceptors, through 5-HT1a auto-receptor is also enhanced in the raphe nucleus. As a result, the neurotransmission in the serotonergic neuron is not enhanced to an expected degree as a whole by initial administration of SSRI. Accordingly, the inventor appreciates that a reduction of a period required for the exhibition of the effect of SSRI or the enhancement of the serotonin effect in combination with an increase in acetylcholine for improving memory and mood can be achieved either by stopping the negative feedback reaction of serotonin by inhibiting the serotonin 1A auto-receptor by the use of a serotonin 1A receptor antagonist, or by reducing a period required for the desensitization by positively stimulating the serotonin 1A auto-receptor by the use of a serotonin 1A receptor agonist. This would similarly apply to other 5HT receptors that affect mood.

As an alternate pathway to modifying negative mood, the inventor has identified that Norepinephrine uptake 2 inhibitors (or their precursors) may be beneficial in the identified combination of an antidepressant and an cholinesterase inhibitor to treat mood and depression. Norepinephrine uptake 2 inhibitors are administered to enhance the effect of norepinephrine reuptake inhibitors and other antidepressants. This would similarly apply to other norepinephrine receptors that affect memory or mood.

The combinational therapy used in the present study includes at least one anti-cholinesterase inhibitor.

Anti-cholinesterase compounds suitable for delivery include Physostigmine, 1, 2, 3,3a,8,8a-hexahydro-1,3a,8-trimethylpyrrolo[2,3-b]indol-5-ol methylcarbamate, or a salt thereof, and structurally similar compounds. As used above, the term “salt thereof is meant to include any nontoxic pharmaceutically suitable salt of a compound described above with the desired pharmacological properties in mammals. Preparation of such a salt is well-known to those skilled in pharmaceutical science.

Pharmaceutically acceptable acid addition salts of the above compounds include: hydrochloride, hydrobromide, hydroiodide, sulfate, bisulfate, nitrate, salicylate, citrate, tartarate, bitartarate, lactate, phosphate, malate, maleate, fumarate, succinate, acetate and pamoate. Acid forms thereof.

Also suitable for buccal/sublingual delivery or administration using other cholinesterase inhibitors such as Metrifonate, Donepezil, and structurally similar compounds would be useful.

A pharmaceutical compound containing an anti-cholinesterase of the present invention to be combined with at least one anti-depressant can be prepared by processes that are known in the art and described, for example, in U.S. Pat. No. 4,895,841, WO 98/39000, and Japanese Patent Application Nos. 4-187674 and 4-21670, the disclosures of each of which are incorporated by reference herein in their entirety. For example, Donepezil hydrochloride, a preferred cholinesterase inhibitor for use in the methods described herein, is commercially available as ARICEPT® from Eisai Inc., Teaneck, N.J.

The dosage regimen for treating the diseases described herein with the cholinesterase inhibitors, as part of the combination treatment for mood and memory described herein is selected in accordance with a variety of factors, including the age, weight, sex, and medical condition of the patient, the severity of the disease affecting mood and memory, the route of administration, pharmacological considerations such as the activity, efficacy, pharmacokinetic and toxicology profiles of the particular cholinesterase inhibitor used and the type of drug delivery system used. Importantly consideration will be given to the combination therapy that includes at least one other anti-depressant that is used in combination with the cholinesterase inhibitor, as well, as the possibility of using a BDNF compound or stimulator and or an anti-depressant enhancer. Thus, the dosage regimen actually used may vary widely and may deviate from the preferred dosage regimen described herein.

In certain embodiments of the present invention, the cholinesterase inhibitors are administered to treat the consequences of memory and mood as a result of diseases or disorders described herein in doses of about 0.1 milligram to about 300 milligrams per day, preferably about 1 milligram to about 100 milligrams per day, more preferably about 5 milligrams to about 10 milligrams per day. The doses can be administered in one to four portions over the course of a day, preferably once a day. One skilled in the art will recognize that when the cholinesterase inhibitors of the present invention are administered to children, the dose may be smaller than the dose administered to adults, and that the dose can be dependent upon the size and weight of the patient. In preferred embodiments, a child can be administered the cholinesterase inhibitors of the present invention in doses of about 0.5 milligrams to about 10 milligrams per day, preferably about 1 milligram to about 3 milligrams per day.

In the methods described herein, a physician can administer patients donepezil hydrochloride, which is commercially available as ARICEPT® (Eisai Inc., Teaneck, N.J.), as film-coated tablets containing 5 milligrams donepezil hydrochloride or 10 milligrams donepezil hydrochloride. The tablets can be administered one to about four times a day. In preferred embodiments, one 5 milligram or one 10 milligram ARICEPT® tablet is administered once a day for the methods described herein to treat mood and memory in combination with at least one anti-depressant. One skilled in the art will appreciate that when donepezil hydrochloride is administered to children, the dose may be smaller than the dose that is administered to adults. In preferred embodiments, a child can be administered donepezil hydrochloride in doses of about 0.5 milligrams to about 10 milligrams per day, preferably about 1 milligram to about 3 milligrams per day.

In general the present invention relates to methods to increase at least 2 neurotransmitters in the brain acetylcholine and at least one of either serotonin and or nor-adrenaline.

It is preferable that antidepressants are used to increase serotonin and or noradrenaline.

In other embodiments of the invention the anti-depressant may also be catechol-O-methyltransferase inhibitor. Compounds with COMT inhibiting activity are already known. For example, derivatives of catechols and isoflavones as COMT inhibitors have been disclosed i.a. in U.S. Pat. No. 5,446,194, U.S. Pat. No. 5,389,653 and, respectively, in U.S. Pat. No. 3,973,608. COMT inhibitors are used i.a. in the treatment of Parkinson's disease. COMT-inhibators have also indicated to be useful in the treatment of i.a. hypertension, heart failure and depression (cf. e.g. U.S. Pat. No. 446,194 above) as well as inhibitors for the prevention of diabetic vascular dysfunctions (cf. WO-A-98 27973).

In other embodiments of the invention the anti-depressant may be selected from classical antidepressants being a tri-cyclic antidepressant (TCA).

In yet other embodiments of the invention an antidepressant that inhibits dopamine reuptake can be used such as Bupropion.

Selective serotonin reuptake inhibitors (hereinafter abbreviated as SSRI in some cases). SSRI has a selective serotonin reuptake inhibitory effect more selective. Specific SSRIs that have utility for the present invention but not limited to, include for example, zimelidine fluoxetine, fluvoxamine, citalopram, cericlamine, femoxetine, ifoxetine, cyanodothiepin, sertraline, paroxetine and lotoxetine.

It is preferable that the serotonin re-uptake inhibitor is selective for serotonin. In another embodiment the drug combination of an antidepressant SSRI and a cholinesterase inhibitor for mood and memory may be combined with a compound having affinity for serotonin IA receptors. An example, of such a compound is pindolol having a high affinity for serotonin IA receptors such that increases the effect of a serotonin reuptake inhibitor in a melancholiac and reduces a period required for the onset of the effect (Arch, Gen. Psychiatry, (1994), 51, 248-251).

Examples of pharmaceutically effective salts for the antidepressant reuptake inhibitors include, but are not limited to salts prepared from pharmaceutically acceptable acids or bases, including organic and inorganic acids and bases. When the preferred compound of use is basic, salts may be prepared from pharmaceutically acceptable acids. Suitable pharmaceutically acceptable acids include acetic, benzenesulfonic (besylate), benzoic, p-bromophenylsulfonic, camphorsulfonic, carbonic, citric, ethanesulfonic, fumaric, gluconic, glutamic, hydrobromic, hydrochloric, hydroiodic, isethionic, lactic, maleic, malic, mandelic, methanesulfonic (mesylate), mucic, nitric, oxalic, pamoic, pantothenic, phosphoric, succinic, sulfuric, tartaric, p-toluenesulfonic, and the like. Examples of such pharmaceutically acceptable salts include, but are not limited to, acetate, benzoate, hydroxybutyrate, bisulfate, bisulfite, bromide, butyne-1,4-dioate, carpoate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, and the like.

In an embodiment of the present invention the anti-depressant is a norepinephrine reuptake inhibitor. The norepinephrine reuptake can be selective for noradrenaline only or alternatively can be non-selective or both.

Examples of norepinephrine reuptake inhibitors that could be used according to the invention with selective norepinephrine reuptake inhibitors being particularly preferred. This list of norepinephrine reuptake inhibitor compounds includes, but is not limited to the following: tandamine, pirandamine, ciclazindol, fluparoxan, lortalamine, talsupram, talopram, prindamine, nomifensine, viloxazine, tomoxetine, duloxetine, venlafaxine, milnacipran and reboxetine.

In a preferred embodiment the selective norepinephrine reuptake inhibitor is reboxetine, 2-[.alpha.-((2-ethoxyphenoxy)benzyl]-morpholine, and its pharmaceutically acceptable salts, in either its enantiomeric (particularly the (S,S) enantiomer) or racemic form. Synthesis of racemic reboxetine is described in greater detail in U.S. Pat. No. 4,229,449. Individual stereoisomers of reboxetine can be obtained by resolution of the racemic mixture of enantiomers using conventional methods generally known by those skilled in the art. Such methods include, but are not limited to, resolution by simple crystallization and chromatographic techniques, for example, as set forth in GB 2,167,407. Other methods of preparation are described in U.S. Pat. Nos. 5,068,433 and 5,391,735. Reboxetine can be a free base form, or it can be in salt form, preferably the methanesulfonate salt (also called reboxetine mesylate).

The selection of the dosage of the antidepressant component of the invention for a nor-adrenaline re-uptake inhibitor is that which can provide relief to the patient. As is well known, the dosage of this component depends on several factors such as the potency of the selected specific compound, the mode of administration, the age and weight of the patient, the severity of the condition to be treated, and the like. This is considered to be within the skill of the artisan and one can review the existing literature on the components to determine optimal dosing. Desirably, when reboxetine is selected as the active agent, the daily dose contains from about 0.1 mg. to about 10 mg. More preferably, each dose of the component contains about 0.5 to about 8 mg of the active ingredient, and even more preferably, each dose contains from about 0.5 to about 5 mg of the active ingredient. This dosage form permits the full daily dosage to be administered in one or two oral doses. This will allow for final formulations containing 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9. 2.0, 2.1, 2.2, 2.3, 2.4, or 2.5 mg of active. More than once daily or twice daily administrations (e.g., 3, 4, 5 or 6 administrations per day) are also expressly contemplated herein.

The average daily adult dosage of the other nor-epinephrine reuptake inhibitors is as follows. The dosages expressly include all numerical values, whole or fractional, within the stated range. Paediatric dosages may be less.

Component Average Daily Dosage (mg/day/patient) Tandamine 7.5 to 3750 Pirandamine 7.5 to 3750 Ciclazindol 5 to 500 Fluparoxan 0.75 to 750 Lortalamine 1 to 200 Talsupram 1 to 3750 Talopram 1 to 3750 Prindamine 1 to 3750 Nomifensine 1 to 80 Viloxazine 1 to 3750 Tomoxetine 1 to 200 Duloxetine 5 to 500 Venlafaxine 2 to 200 Milnacipran 7.5 to 75.

In another embodiment the norepinephrine uptake 2 inhibitors may also be combined with MAO inhibitors or with selective serotonin reuptake inhibitors in combination with a cholinesterase inhibitor.

In another embodiment the norepinephrine reuptake inhibitor may be combined with a norepinephrine reuptake uptake 2 inhibitor. The formulation may be combined into a single medication with a norepinephrine reuptake inhibitor, such as imipramine, desipramine, or reboxetine, in order to inhibit both uptake mechanisms.

Alternatively, the norepinephrine uptake 2 inhibitors may be useful antidepressants in their own right, without the need for co-administration of other antidepressants and used in combination with acytlcholinesterase.

One class of norepinephrine uptake 2 inhibitors is normetanephrine (the O-methylated metabolite of norepinephrine) and normetanephrine precursors [such as 3(4-hydroxy-3-methoxyphenyl)-serine (4H-3MePS), particularly L-threo-3-(4-H-3 MePS)] that are transported to the brain where they are converted into normetanephrine, thereby enhancing the effect of other antidepressants. For example, the invention enhances the antidepressant effect of norepinephrine reuptake inhibitors and cholinesterase inhibitors to improve and or stabilize and or modulate and or treat mood and memory within the same individual.

In one preferred embodiment the anti-depressant is a serotonin reuptake inhibitor. The serotonin reuptake inhibitor can be selective for serotonin only or alternatively can be non-selective or both. In a further embodiment of the invention BDNF levels are modulated to improve memory and mood within the same individual when they are co-treated with an anti-cholinesterase drug and an antidepressant.

In a further embodiment of the invention BDNF expression is up-regulated in the brain. Preferably BDNF levels are up-regulated in the hippocampus and or within other areas of the brain that affect memory and mood.

Preferably an increase in BDNF will also be positively influenced via the use of the said composition that includes both an antidepressant and a method to increase brain acytlcholine in affected areas that positively influence mood and memory.

In yet another embodiment of the invention exercise is used to up-regulate BDNF. BDNF can also enhance serotonergic effects.

In other embodiment of the invention anti-depressants are used to up-regulate BDNF.

In yet other embodiments of the invention increased levels of nor-adrenaline are used to increase BDNF and phosphorylated Trk.

In other embodiments of the invention ERK and PI-3K inhibitors are used to promote and or remove negative feedback mechanisms that interfere with the up-regulation of BDNF.

In other embodiments of the invention BDNF the compound may be transported to the brain such that BDNF is conjugated to a blood-brain barrier (BBB) molecular Trojan horse. The latter may be a peptidomimetic monoclonal antibody (MAb) to the transferrin receptor—see Zhang Y. Pardridge WM. Blood-brain barrier targeting of BDNF improves motor function in rats with middle cerebral artery occlusion.Brain Res. 2006 Jul. 31.

In certain embodiments of the invention oestrogen promoting substances can be used to up-regulate BDNF.

The BDNF used in the present invention may be any one of any animal origin, such as mouse, pig, or human, and can be prepared by various processes. When a BDNF isolated from animal tissues is used in the present invention, it may be purified to such a degree that it can be used as a medicament (cf., The EMBO Journal, 5, 549-553 (1982)). Alternatively, a BDNF can be obtained by culruring a primary culture cell or an established cell line which can produce BDNF, and isolating from the culture broth. Moreover, there may be used a recombinant BDNF which can be obtained by a conventional gene engineering technique, e.g., by inserting a gene coding for BDNF into a suitable vector, transforming a suitable host with the recombinant vector, and isolating from the culture supernatant of the resulting transformant (cf., Proc. Natl. Acad. Sci. U.S.A., 88, 961 (1991); Biochem. Biophys. Res. Commun., 186, 1553 (1992)), which is suitable for production of BDNF of uniform property in a large scale. The host cells to be used in the above process are not critical, and may be any conventional host cells which have been used in gene engineering techniques, for example, Escherichia coli, Bacillus subtilis, yeasts, vegetable cells or animal cells.

A stable pharmaceutical composition of brain derived neurotrophic factor (BDNF) in the form of an aqueous solution or lyophilized one being suitable for a long-term storage, which contains a surfactant, especially nonionic surfactant (e.g., Tween 80) of 0.001 to 10%, whereby the polymerization and the denaturation of BDNF are inhibited, and the biological activities of BDNF are maintained for a long time. The lyophilized composition can be made more stable by addition of a sugar alcohol (e.g., mannitol) and/or an amino acid (e.g., glycine). The description for the composition of this active form of BDNF can be found in U.S. Pat. No. 6,077,829.

Compositions of the present invention can conveniently be administered in a pharmaceutical composition containing the active components in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). To the extent necessary for completion, this reference is hereby incorporated by reference. The compositions of the present invention can be administered parenterally (for example, by intravenous, intraperitoneal or intramuscular injection), topically, orally, intranasally, intravaginally, or rectally, with oral administration being particularly preferred.

For oral therapeutic administration, the inventive composition may be combined with one or more excipients and used in the form of ingestible tablets, buccal tablets, troches, capsules, elixirs, suspensions, syrups, wafers, chewing gums, foods and the like. Such compositions and preparations should contain at least 0.1% of active compound. The percentage of the compositions and preparations may, of course, be varied and may conveniently be between about 0.1 to about 100% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it may contain, in addition to materials of the above type, a liquid carrier, such as a vegetable oil or a polyethylene glycol.

Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active components may be incorporated into sustained-release preparations and devices including, but not limited to, those relying on osmotic pressures to obtain a desired release profile.

The inventive composition, containing the two active components, may be administered in the same physical form or concomitantly according to the above-described dosages and in the above-described delivery vehicles. The dosages for each active component can be measured separately and can be given as a single combined dose or given separately. They may be given at the same or at different times as long as both actives are in the patient at one time over a 24-hour period. Concomitant or concurrent administration means the patient takes one drug within about 5 minutes of taking the other drug. Because the goal is to provide rapid symptomatic relief to the patient, in most cases when treatment is started the two drugs would be administered to the patient close in time and typically concomitantly; thereafter, the timing of each drug's administration may not be as important.

Although the invention has been described by way of particular embodiments it will be understood that variations in keeping with the disclosure and the spirit of the invention described are also within its scope.

What is claimed is:

1. Method of prophylactic or therapeutic treatment of memory and mood in a subject comprising the administration of a therapeutically effective amount of at least one memory enhancing agent and at least one antidepressant to a subject requiring such treatment.

2. Method according to claim 1, comprising the administration of a therapeutically effective amount of at least one antidepressant and at least one cholinesterase inhibitor to a subject requiring such treatment.

3. Method according to claim 1, comprising the administration of a therapeutically effective amount of at least one cholinesterase inhibitor and at least one serotonin uptake or reuptake inhibitor to a subject requiring such treatment.

4. Method according to claim 1, comprising the administration of a therapeutically effective amount of at least one anti-cholinesterase agent and at least one norepinephrine reuptake inhibitor to a subject requiring such treatment.

5. Method according to claim 1, further comprising the administration of one or more nor-adrenaline reuptake inhibitors.

6. Method according to claim 1, further comprising the administration of a stimulator and/or inducer of BDNF expression and/or activity.

7. Method according to claim 6, wherein the anti-depressant up-regulates the expression of BDNF.

8. Method according to claim 1, further comprising the administration of a 5HT1a agonist or antagonist.

9. Method of prophylactic or therapeutic treatment of memory and mood comprising the administration of a therapeutically effective amount of at least one anti-cholinesterase agent and one or more of the following: (a) a 5HT1 agonist or antagonist, (b) a compound that increases BDNF expression or activity, (c) an anti-depressant, to a subject requiring such treatment.

10. The method of claim 1, wherein the antidepressant is a norepinephrine uptake inhibitor.

11. The method of claim 1, wherein the norepinephrine uptake inhibitor is selected from Reboxetine, Pirandamine, Ciclazindol, Fluparoxan, Lortalamine, Talsupram, Talopram, Prindamine, Nomifensine, Viloxazine, Tomoxetine, Duloxetine, Venlafaxine, Milnacipran.

12. The method of claim 1, wherein the antidepressant is a norepinephrine uptake 2 inhibitor that enhances norepinephrine reuptake inhibitors and or other antidepressants.

13. The method of claim 1, wherein SSRI anti-depressant efficacy is increased via the use of a 5HT1 antagonist.

14. The method of claim 6, wherein BDNF is a recombinant protein.

15. The method of claim 6, wherein BDNF activity is increased via the use of anti-depressant.

16. The method of claim 6, wherein BDNF activity is increased via the use of an oestrogen.

17. The method of claim 6, wherein BDNF activity is increased by agents capable of increasing the expression of BDNF.

18. The method of claim 1, wherein the cholinesterase inhibitor is selected from Metrifonate and Donepezil.

19. The method of claim 1, wherein the at least one memory enhancing agent and the at least one anti-depressant are administered simultaneously.

20. The method of claim 1, wherein the at least one memory enhancing agent and the at least one anti-depressant are administered consecutively.
